(19) United States
(12) Patent Application Publication
(10) Pub. No.: US 2026/0357305 A1
(43) Pub. Date: Nov. 12, 2026

(54) FRUCTOSE DECOMPOSITION METHOD, FRUCTOSE DECOMPOSITION DEVICE, MANNITOL PRODUCTION METHOD, AND MANNITOL PRODUCTION DEVICE

(71) Applicant: Panasonic Intellectual Property Management Co., Ltd., Osaka (JP)

(72) Inventors: NORIYUKI HATSUGAI, Kyoto (JP); DAISUKE KOYA, Osaka (JP); FUMIYA WAYAMA, Osaka (JP)

(21) Appl. No.: 19/717,713

(22) Filed: Jun. 24, 2026

Related U.S. Application Data
(63) Continuation of application No. PCT/JP2024/045394, filed on Dec. 23, 2024.

(30) Foreign Application Priority Data
Jan. 17, 2024 (JP) 2024-005349

(51) Int. Cl. C12P 7/18; C12N 9/04; C25B 3/09; C25B 3/25

(52) U.S. Cl. CPC C12P 7/18; C12N 9/0006; C12Y 101/01; C25B 3/09; C25B 3/25

(57) ABSTRACT

A fructose decomposition method according to the present disclosure includes bringing a liquid containing mannitol dehydrogenase, fructose, and a coenzyme into contact with a working electrode to electrochemically reduce the coenzyme and decompose the fructose by the mannitol dehydrogenase.

### BACKGROUND

### 1. Technical Field

The present disclosure relates to a fructose decomposition method, a fructose decomposition device, a mannitol production method, and a mannitol production device.

### 2. Description of the Related Art

Conversion of sugars such as fructose and glucose by using enzymes and coenzymes is known.

For example, Japanese Examined Patent Application Publication No. 63-14301 discloses an enzymatic electrode in which D-mannitol dehydrogenase (DMDH) is immobilized on a current collector. This enzymatic electrode is used in analyzing the concentration of fructose contained in food. For example, in a measurement system equipped with this enzymatic electrode, reduced nicotinamide adenine dinucleotide (NADH) is dissolved as a coenzyme in a buffer solution, and the potential of the enzymatic electrode is set to −1.2 V with respect to a reference electrode. In this case, adding fructose to the buffer solution increases the cathodic current. The increase in the cathodic current is based on the reaction of reducing fructose by the action of DMDH and reducing NAD⁺, which has been generated by oxidation of NADH in coupling with the reduction of fructose, to its original reduced form. It is possible to measure the fructose concentration by electrochemically measuring NADH, concentration of which decreases in coupling with the enzymatic reaction.

International Publication No. 2023/074453 discloses an electrochemical reaction device. This electrochemical reaction device includes a first tank, a second tank, a membrane, and a voltage application device. The first tank accommodates glucose dehydrogenase and nicotinamide adenine dinucleotide (NAD⁺), and has a working electrode placed therein. In this electrochemical reaction device, glucose dehydrogenase functions as a catalyst and NAD⁺ is involved as a coenzyme to decompose glucose and generate gluconolactone. Along with this reaction, NAD⁺ turns into reduced nicotinamide adenine dinucleotide (NADH). When the voltage application device applies voltage between the working electrode and the counter electrode, the NADH generated by the aforementioned reaction is oxidized by the working electrode, and NAD⁺ is generated. In this electrochemical reaction device, since NADH is continuously oxidized and NAD⁺ is generated, it is possible to keep decomposing glucose without using large quantities of nicotinamide adenine dinucleotide.

### SUMMARY

In one general aspect, the techniques disclosed here feature a fructose decomposition method including bringing a liquid containing mannitol dehydrogenase, fructose, and a coenzyme into contact with a working electrode to electrochemically reduce the coenzyme and decompose the fructose by the mannitol dehydrogenase.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic view of one example of a fructose decomposition device according to an embodiment;

FIG. 2 is a functional block diagram illustrating the fructose decomposition device according to an embodiment;

FIG. 3 is a schematic diagram of a fructose decomposition mechanism;

FIG. 4 is a graph showing the relationship between the current value of the working electrode and the voltage application time in fructose decomposition;

FIG. 5 is a flowchart illustrating one example of a fructose decomposition method according to an embodiment;

FIG. 6 is a graph showing changes in fructose amount in Example 1; and

FIG. 7 is a graph showing changes in fructose amount in Comparative Example 1.

### DETAILED DESCRIPTIONS

The features disclosed in Japanese Examined Patent Application Publication No. 63-14301 and International Publication No. 2023/074453 leave room for reconsideration from the viewpoint of decomposing fructose by enzymatic reaction. To address this, the present disclosure provides advantageous features from the viewpoint of decomposing fructose by enzymatic reaction.

A fructose decomposition method of the present disclosure is advantageous from the viewpoint of decomposing fructose by enzymatic reaction.

Underlying Knowledge Forming Basis of the Present Disclosure

In recent years, excessive intake of fructose has been linked with obesity and some cancers. Thus, it is anticipated that the demand for the features that efficiently turn fructose into sugar alcohols or the like will increase. Mannitol dehydrogenase (MDH), for example, functions as a catalyst in the reaction of formula (1) below in which reduced nicotinamide adenine dinucleotide (NADH) is involved as a coenzyme.

D-Fructose+NADH+H⁺→D-mannitol+NAD⁺ Formula (1)

As disclosed in Japanese Examined Patent Application Publication No. 63-14301, when the fructose concentration in a particular sample is to be measured through the aforementioned reaction, for example, changes in NADH concentration coupled with this reaction can be measured. In such a case, it is considered that the amount of NADH consumed by the fructose concentration measurement is not that large. Meanwhile, in converting fructose into mannitol in a fructose-containing liquid through the aforementioned reaction, there are instances where it is difficult to add a coenzyme, such as NADH, in a concentration corresponding to the amount of fructose contained in the liquid. This is because there is an upper limit to the concentration of NADH dissolved in a liquid. Thus, it is not easy to decompose fructose by enzymatic reaction.

The inventors of the present disclosure have conducted extensive studies to address the aforementioned issue. As a result, it has been discovered that the coenzyme can be regenerated into a state suitable for fructose decomposition when an oxidized coenzyme generated in coupling with the catalytic action of the MDH is continuously and electrochemically reduced. As a result, fructose can be decomposed by adding a small amount of a coenzyme. Based on this new finding, the inventors of the present disclosure have arrived at a fructose decomposition method, a fructose decomposition device, a mannitol production method, and a mannitol production device of the present disclosure. Note here that in the present disclosure, “continuously” means “repeatedly”.

### Embodiments of the Present Disclosure

Embodiments of the present disclosure will now be described with reference to the drawings. The embodiments described below are all comprehensive or specific examples. The numerical values, shapes, materials, constituting elements, the positional arrangement and connection forms of the constituting elements, steps, and the order of steps indicated in the following embodiments are merely examples and are not intended to limit the scope of the claims. Moreover, the drawings are not necessarily faithful. In the drawings, substantially identical structures are represented by the same reference signs, and the descriptions therefor may be omitted or simplified to avoid redundancy. In the description below, the terms that indicate relationships between elements, such as parallel and perpendicular, the terms that indicate the shapes of the elements, such as rectangular, and the numerical ranges not only have the strict meanings but also include substantially equivalent ranges, for example, differences of about several percentage points. These embodiments that are presented as comprehensive or specific examples may be realized through systems, devices, integrated circuits, computer programs, or computer-readable recording media such as CD-ROMs, or through any combination of systems, methods, integrated circuits, computer programs, and recording media.

### Embodiments

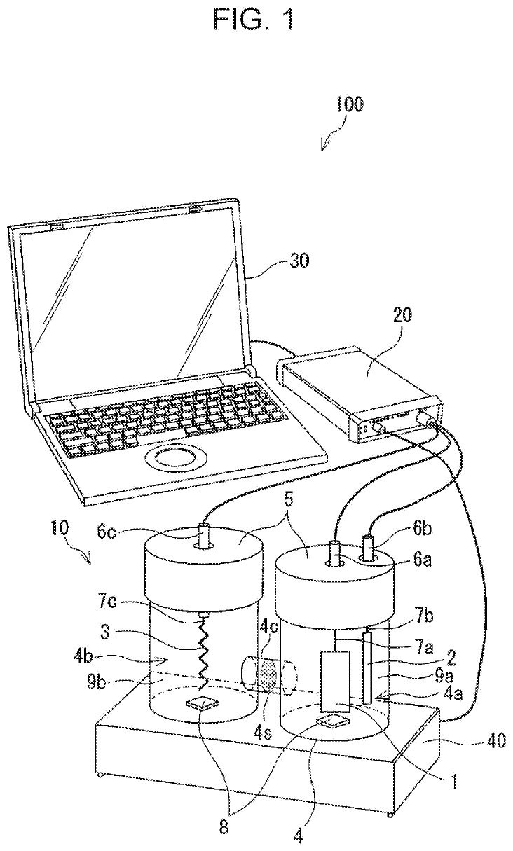

Embodiments will now be specifically described with reference to FIGS. 1 to 5.

FIG. 1 is a schematic view of one example of a fructose decomposition device according to an embodiment. As illustrated in FIG. 1, a fructose decomposition device 100 includes a working electrode 1, a counter electrode 3, and a voltage application device 20. The voltage application device 20 is for applying voltage between the working electrode 1 and the counter electrode 3. The decomposition device 100 electrochemically and continuously reduces a coenzyme by applying voltage between the working electrode 1 and the counter electrode 3 to thereby decompose fructose with mannitol dehydrogenase. It is considered that fructose is converted into mannitol through decomposition. Thus, the decomposition device 100 can be referred to as a mannitol production device. Fructose is, for example, D-fructose, and mannitol is, for example, D-mannitol.

By using the decomposition device 100, for example, a liquid 9a containing mannitol dehydrogenase, fructose, and a coenzyme can be brought into contact with the working electrode 1 to electrochemically and continuously reduce the coenzyme. In addition, fructose can be decomposed by mannitol dehydrogenase. As mentioned above, it is considered that fructose is converted into mannitol through decomposition. Thus, this method can be understood as a method for producing mannitol from fructose through enzymatic reaction.

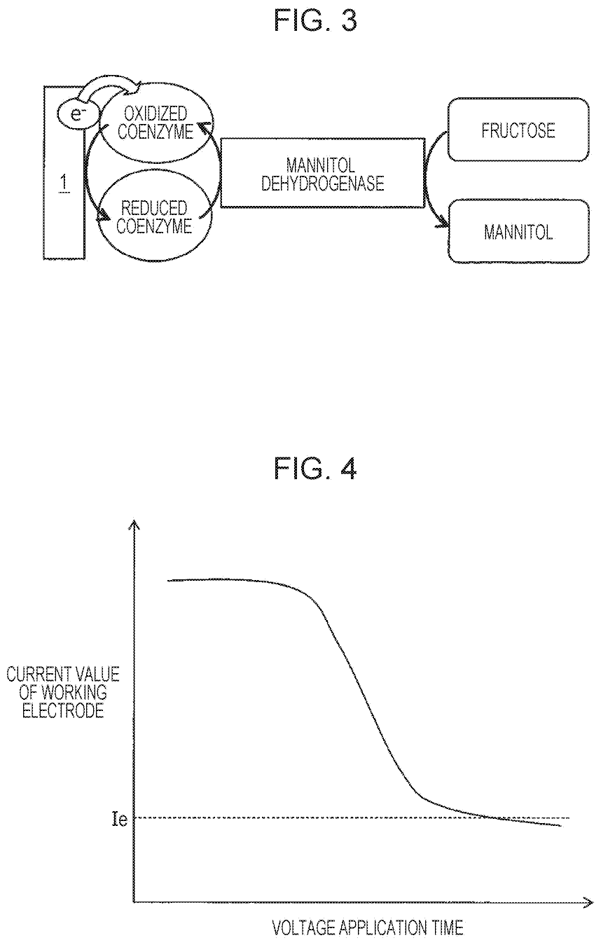

The coenzyme is, for example, oxidized as mannitol is generated from fructose by mannitol dehydrogenase, and is reduced by receiving electrons from the working electrode 1. FIG. 3 is a schematic diagram of a fructose decomposition mechanism. As illustrated in FIG. 3, as mannitol is generated from fructose by mannitol dehydrogenase, a reduced coenzyme turns into an oxidized coenzyme. In addition, the oxidized coenzyme receives electrons from the working electrode 1 and turns into a reduced coenzyme. Fructose is decomposed by repetition of these changes. Thus, for example, it is possible to decrease the concentration of fructose contained in the liquid 9a to a particular value or lower.

In the decomposition device 100, for example, voltage is applied between the working electrode 1 and the counter electrode 3 for a particular period of time. For example, the period for which voltage is applied between the working electrode 1 and the counter electrode 3 is adjusted according to the amount of the liquid 9a and the initial concentration of fructose in the liquid 9a. The larger the amount of the liquid 9a or the higher the initial concentration of fructose in the liquid 9a, the longer the period for which voltage is applied between the working electrode 1 and the counter electrode 3.

In the decomposition device 100, for example, voltage may be applied to the working electrode 1 until the current value in the working electrode 1 reaches a particular value Ie or lower. FIG. 4 is a graph showing the relationship between the current value of the working electrode 1 and the voltage application time in fructose decomposition. As shown in FIG. 4, the longer the voltage application time, the smaller the current value in the working electrode 1. When the concentration of fructose contained in the liquid 9a decreases as a result of conversion of fructose to mannitol, the enzymatic reaction is inhibited, and so is the change from the reduced coenzyme to the oxidized coenzyme. Thus, the amount of the oxidized coenzyme that receives electrons from the working electrode 1 decreases, and the current value in the working electrode 1 decreases. In other words, the fact that the current value in the working electrode 1 is small suggests that the concentration of fructose has decreased.

As long as fructose can be converted into mannitol by mannitol dehydrogenase in the decomposition device 100, the coenzyme is not limited to a particular coenzyme. The coenzyme is, for example, nicotinamide adenine dinucleotide (NADH). In such a case, as mannitol is generated from fructose by mannitol dehydrogenase, reduced NADH is oxidized and oxidized nicotinamide adenine dinucleotide (NAD⁺) is generated. Moreover, oxidized NAD⁺ is reduced by receiving electrons from the working electrode 1, and reduced NADH is generated. The coenzyme may be nicotinamide adenine dinucleotide phosphate (NADPH).

As illustrated in FIG. 1, the decomposition device 100 further includes, for example, a reference electrode 2, a cell 4, covers 5, terminals 6a, 6b, and 6c, leads 7a, 7b, and 7c, stir bars 8, a controller 30, and a stirrer 40. The working electrode 1, the reference electrode 2, the counter electrode 3, the cell 4, the covers 5, the terminals 6a, 6b, and 6c, and the leads 7a, 7b, and 7c constitute a three-electrode-cell electrochemical reactor 10. The electrochemical reactor 10 may be configured as a two-electrode cell that includes a working electrode 1 and a counter electrode 3 but without a reference electrode 2.

The cell 4 has, for example, a first space 4a and a second space 4b inside. The working electrode 1 and the reference electrode 2 are placed in the first space 4a, and the counter electrode 3 is placed in the second space 4b. The cell 4 includes, for example, a connecting part 4c. The connecting part 4c allows the first space 4a and the second space 4b to be in communication with each other, and a separator 4s is placed in the connecting part 4c. The separator 4s separates the space in the connecting part 4c into a space on the first space 4a side and a space on the second space 4b side.

A liquid 9a containing mannitol dehydrogenase, fructose, and a coenzyme can be contained in the first space 4a. In this manner, the liquid 9a can make contact with the working electrode 1. A liquid 9b, for example, can be contained in the second space 4b. In this manner, the liquid 9b can make contact with the counter electrode 3. The liquid 9b is, for example, an electrolyte solution such as a phosphate buffer solution.

The separator 4s, for example, has ion conductivity and blocks penetration of some of the components contained in the liquid 9a and the liquid 9b. For example, the separator 4s blocks penetration of mannitol dehydrogenase, fructose, and the coenzyme contained in the liquid 9a. The separator 4s has, for example, proton conductivity. The separator 4s contains, for example, a polymer having a perfluoro side chain containing a sulfonic acid group.

The surfaces of the working electrode 1 and the counter electrode 3 contain, for example, a conductive material. Examples of the conductive material include carbon materials, conductive polymers, semiconductor materials, or metal materials. The conductive material is, for example, a material that remains undecomposed as the conductive material is oxidized. Examples of the conductive material include carbon materials, conductive polymers, semiconductor materials, and metal materials. Examples of the carbon materials include carbon nanotubes, Ketjenblack (registered trademark), glassy carbon, graphene, fullerenes, carbon fibers, carbon fabric, and carbon aerogel. Examples of the conductive polymers include polyaniline, polyacetylene, polypyrrole, poly(3,4-ethylenedioxythiophene), poly(p-phenylene vinylene), polythiophene, and poly(p-phenylene sulfide). Examples of the semiconductor materials include silicon, germanium, indium tin oxide (ITO), titanium oxide, copper oxide, and silver oxide. Examples of the metal materials include gold, platinum, silver, titanium, aluminum, tungsten, copper, iron, and palladium. The working electrode 1 may be a glassy carbon electrode, and the counter electrode 3 may be a platinum electrode.

In the decomposition device 100, for example, the working electrode 1 functions as a cathode and the counter electrode 3 functions as an anode. When voltage is applied between the working electrode 1 and the counter electrode 3, the potential of the working electrode 1 is, for example, adjusted to a potential at which the oxidized coenzyme can be reduced. The surface area of the counter electrode 3 is, for example, larger than the surface area of the working electrode 1. According to this structure, the reaction rate of the reaction of electrochemically reducing the coenzyme is easily increased. Thus, a large amount of mannitol is easily generated in a short time from fructose contained in the liquid 9a.

The reference electrode 2 does not react with the components contained in the liquid 9a and is maintained at a specified potential. The reference electrode 2 is, for example, a silver/silver chloride electrode.

In the electrochemical reactor 10, the cell 4 is closed by, for example, the covers 5. The terminals 6a, 6b, and 6c are attached to the covers 5. The lead 7a extends from the terminal 6a and electrically connects the terminal 6a to the working electrode 1. The lead 7b extends from the terminal 6b and electrically connects the terminal 6b to the reference electrode 2. The lead 7c extends from the terminal 6c and electrically connects the terminal 6c to the counter electrode 3. The terminals 6a, 6b, and 6c are electrically connected to the voltage application device 20.

The voltage application device 20 applies, for example, voltage between the working electrode 1 and the counter electrode 3 according to the control signal output from the controller 30 to adjust the potential difference between the working electrode 1 and the reference electrode 2 to a particular value.

The controller 30 processes information to control the voltage application by the voltage application device 20 and the movement of the motor (not illustrated) of the stirrer 40. The controller 30 includes, for example, a memory storing a program for controlling the voltage application by the voltage application device 20 and the movement of the motor of the stirrer 40, and an arithmetic device, such as a processor, that reads out the program and performs a particular computation.

In the stirrer 40, the movement of the motor is controlled according to the control signal output from the controller 30. In this manner, the rotation rate and the rotation time of the stir bars 8 respectively placed in the first space 4a and the second space 4b are adjusted.

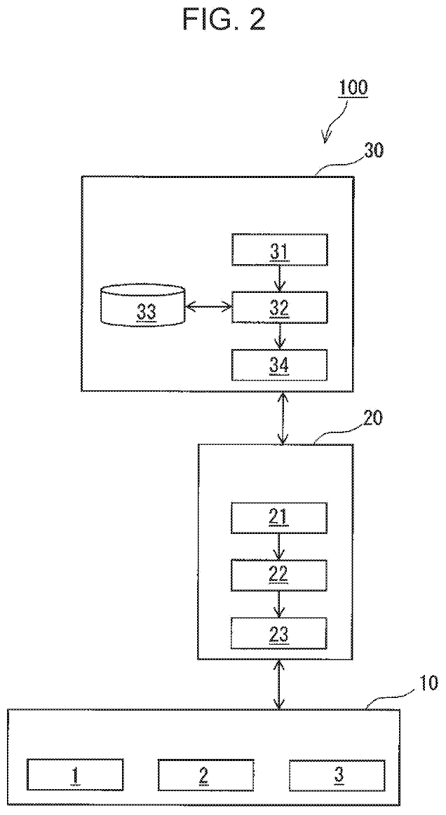

FIG. 2 is a functional block diagram illustrating the decomposition device 100. As illustrated in FIG. 2, the voltage application device 20 includes, for example, an acquisition unit 21, an information processing unit 22, and a voltage control unit 23.

The acquisition unit 21 acquires a control signal output from the controller 30. The acquisition unit 21 may also acquire the potentials of the electrodes in the electrochemical reactor 10, and the measurement data such as the current value flowing in the liquid 9b.

The information processing unit 22 processes information acquired by the acquisition unit 21. For example, upon receiving a control signal from the acquisition unit 21, the information processing unit 22 outputs the acquired control signal to the voltage control unit 23. When the voltage control unit 23 starts to apply voltage to each of the electrodes of the electrochemical reactor 10, the information processing unit 22 acquires the potentials of the respective electrodes of the electrochemical reactor 10 acquired from the acquisition unit 21 and measurement data such as the current value flowing in the liquid 9b. The information processing unit 22 uses the acquired data as the basis for calculating the voltage applied to the working electrode 1 such that the potential difference between the working electrode 1 and the reference electrode 2 is maintained at a particular value. Then a control signal that adjusts the voltage of the working electrode 1 to the calculated voltage is output to the voltage control unit 23.

The voltage control unit 23 applies voltage to each of the electrodes of the electrochemical reactor 10 on the basis of the control signal output from the information processing unit 22.

The voltage application device 20 and the controller 30 may be separate as illustrated in FIG. 1 or may be integrated.

The controller 30 includes, for example, an acquisition unit 31, an information processing unit 32, a storage unit 33, and an output unit 34.

The acquisition unit 31 acquires, for example, instruction information which is information regarding instructions input by the user, and outputs the acquired instruction information to the information processing unit 32.

The information processing unit 32 determines, for example, the conditions under which voltage is applied to each of the electrodes of the electrochemical reactor 10 on the basis of the instruction information acquired by the acquisition unit 31. The instruction information includes, for example, the type of enzyme, the amount of the liquid 9a, the time taken for the mannitol-generating reaction to complete, the clock time of the completion of the reaction, etc. The instruction information may include the ratio of the target concentration of the fructose to the initial concentration of the fructose in the liquid 9a or information corresponding to that ratio.

The information processing unit 32 may output to the output unit 34 a control signal that controls voltage application under conditions determined on the basis of the instruction information or may output to the output unit 34 a control signal that controls voltage application under voltage application conditions pre-set by the user.

The output unit 34 outputs the control signal acquired from the information processing unit 32 to the voltage application device 20.

The storage unit 33 stores data such as the instruction information acquired by the acquisition unit 31, computer programs executed by the controller 30 (for example, application programs for controlling the voltage application device 20), etc.

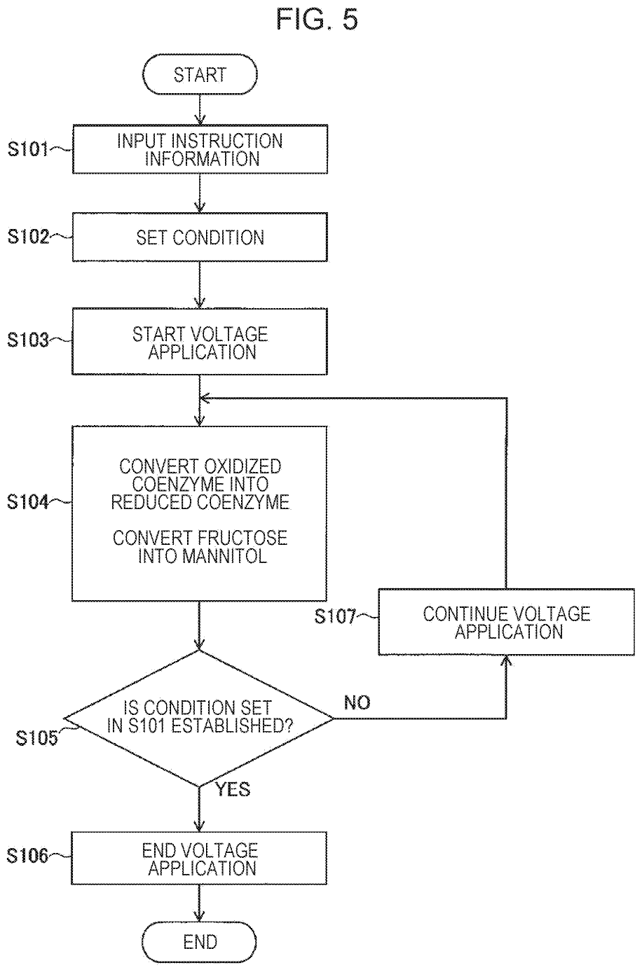

FIG. 5 is a flowchart illustrating one example of the fructose decomposition method. For example, the liquid 9a and the liquid 9b are placed in the cell 4. Next, instruction information is input to the decomposition device 100 in step S101. The instruction information includes, for example, the amount of the liquid 9a, the time taken for the mannitol-generating reaction to complete, the clock time of the completion of the reaction, the ratio of the target concentration of the fructose to the initial concentration of the fructose in the liquid 9a or information corresponding to that ratio, etc.

Next, in step S102, a condition for ending mannitol generation is set on the basis of the instruction information. The condition is, for example, that a particular time has elapsed from the start of voltage application or that the current in the working electrode 1 has reached a particular value Ie or lower. In step S102, conditions related to the target value of the potential of the working electrode 1, the target value of the voltage between the working electrode 1 and the counter electrode 3, and the rotation rate of the stirrer 40 can be set.

Next, in step S103, voltage application is started. For example, the controller 30 generates a control signal according to the target value of the potential of the working electrode 1 and the target value of the voltage between the working electrode 1 and the counter electrode 3. The voltage application device 20 applies voltage between the working electrode 1 and the counter electrode 3 according to the control signal acquired from the controller 30.

Next, in step S104, the oxidized coenzyme is converted into a reduced coenzyme, and fructose is converted into mannitol by the enzymatic reaction involving mannitol dehydrogenase.

Next, the process proceeds to step S105 to determine whether or not the condition set in step S102 for ending mannitol generation is established. When the result of the determination in step S105 is NO, the process proceeds to step S107 to continue applying voltage between the working electrode 1 and the counter electrode 3, and step S104 and step S105 are repeated.

When the determination in step S105 is YES, the process proceeds to step S106, and application of voltage by the voltage application device 20 ends. Then the liquid 9a is recovered, and a series of processes comes to an end.

### Other Embodiments

The fructose decomposition method, the fructose decomposition device, the mannitol production method, and the mannitol production device have been described heretofore through embodiments. However, the present disclosure is not limited to these embodiments. The scope of the present disclosure includes various modifications conceived of by a person skilled in the art and applied to the embodiments, and other embodiments configured by combining some of the constituent elements of the embodiments without departing from the gist of the present disclosure.

### Appendix

The description heretofore discloses the following features.

### Feature 1

A fructose decomposition method including:
bringing a liquid containing mannitol dehydrogenase, fructose, and a coenzyme into contact with a working electrode to electrochemically reduce the coenzyme and decompose the fructose by the mannitol dehydrogenase.

### Feature 2

The fructose decomposition method described in Feature 1,
in which the coenzyme is oxidized as mannitol is generated from the fructose by the mannitol dehydrogenase, and the coenzyme is reduced by receiving electrons from the working electrode.

### Feature 3

The fructose decomposition method described in Feature 1 or 2,
in which the coenzyme is nicotinamide adenine dinucleotide.

### Feature 4

The fructose decomposition method described in any one of Features 1 to 3,
in which voltage is applied to the working electrode for a particular period of time.

### Feature 5

The fructose decomposition method described in any one of Features 1 to 4,
in which voltage is applied to the working electrode until a current value in the working electrode has reached a particular value or lower.

### Feature 6

A fructose decomposition device including:
a working electrode;
a counter electrode; and
a voltage application device for applying voltage between the working electrode and the counter electrode,
in which the fructose decomposition device electrochemically reduces a coenzyme by applying the voltage and decomposes fructose by mannitol dehydrogenase.

### Feature 7

The fructose decomposition device described in Feature 6,
in which the working electrode supplies electrons to the coenzyme by applying the voltage.

### Feature 8

The fructose decomposition device described in Feature 6 or 7,
in which the coenzyme is nicotinamide adenine dinucleotide.

### Feature 9

The fructose decomposition device described in any one of Features 6 to 8,
in which the voltage application device keeps applying the voltage for a particular period of time.

### Feature 10

The fructose decomposition device described in any one of Features 6 to 9,
in which the voltage application device keeps applying the voltage until a current value in the working electrode has reached a particular value or lower.

### Feature 11

A mannitol production method including:
bringing a liquid containing mannitol dehydrogenase, fructose, and a coenzyme into contact with a working electrode to electrochemically reduce the coenzyme and convert the fructose into mannitol by the mannitol dehydrogenase.

### Feature 12

A mannitol production device including:
a working electrode;
a counter electrode; and
a voltage application device for applying voltage between the working electrode and the counter electrode,
in which the mannitol production device electrochemically reduces a coenzyme by applying the voltage and converts fructose into mannitol by mannitol dehydrogenase.

### EXAMPLES

The present disclosure will now be described in further detail through examples. The present disclosure is not limited to the examples below.

### Example 1

A device corresponding to the decomposition device 100 illustrated in FIG. 1 was prepared. A platinum plate having an area of 1.25 cm² in a plan view was used as the working electrode. A silver/silver chloride (Ag/AgCl) electrode was used as the reference electrode. An electrode in which a platinum wire having a wire diameter of 0.5 mm and a length of 23 cm was wound into a coil was used as the counter electrode. The surface area of the counter electrode was larger than the surface area of the working electrode. As the voltage application device, potentiostat OctoStat30 available from Ivium Technologies B.V. was used. The working electrode, the reference electrode, and the counter electrode were respectively electrically connected to a working electrode terminal, a reference electrode terminal, and a counter electrode terminal of the potentiostat to configure a three-electrode cell. An ion exchange membrane, Nafion 117, available from MTI Corporation, was used as the membrane separating the space inside the three-electrode cell in which the working electrode and reference electrode were placed from the space inside the three-electrode cell in which the counter electrode was placed. Nafion is a registered trademark.

A phosphate buffer solution (PBS) having a pH of 7.4 available from Nippon Gene Co., Ltd., was placed in a space inside the three-electrode cell where the counter electrode was placed. In this space, a portion of the counter electrode was in contact with the PBS. The amount of the PBS placed in the space where the counter electrode was placed was 20 mL.

To a separately prepared PBS described above, fructose, reduced NADH, and mannitol dehydrogenase were added to prepare a raw material solution of Example 1. As fructose, D(+)-fructose available from Fujifilm Wako Pure Chemical Industries, Ltd., was used. As reduced NADH, β-nicotinamide adenine dinucleotide, reduced disodium salt available from Sigma-Aldrich was used. As mannitol dehydrogenase, mannitol dehydrogenase (MDH) available from Megazyme Limited was used. The fructose concentration in the raw material solution of Example 1 was 350 mM. The reduced NADH concentration in the raw material solution of Example 1 was 5.6 mM. The MDH concentration in the raw material solution of Example 1 was 2 unit/mL. Into a space inside the three-electrode cell where the working electrode and the reference electrode were placed, 20 mL of the raw material solution of Example 1 was placed. The raw material solution of Example 1 was in contact with a portion of the working electrode and a portion of the reference electrode.

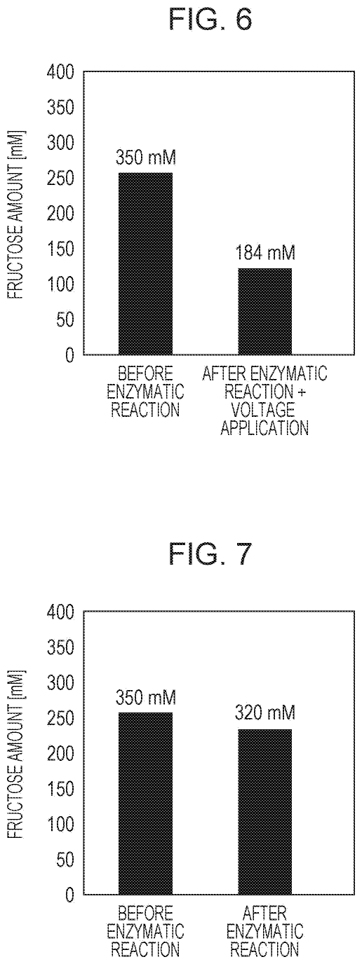

While the PBS placed in the space where the counter electrode was placed and the raw material solution of Example 1 were each stirred with a magnetic stirrer, a particular voltage was applied between the working electrode and the counter electrode at a temperature in the range of 20° C. to 25° C. for 16 hours. As a result, a treated solution of Example 1 was obtained from the raw material solution of Example 1. The fructose concentration in the treated solution of Example 1 was measured with Fructose Colorimetric/Fluorometric Assay Kit available from Sigma-Aldrich. The result is shown in FIG. 6.

FIG. 6 is a graph showing changes in fructose amount in Example 1. In FIG. 6, the vertical axis indicates the fructose concentration. As shown in FIG. 6, the fructose concentration in the treated solution of Example 1 was 184 mM. It is considered that 48% of fructose in the raw material solution of Example 1 was converted due to the enzymatic reaction.

### Comparative Example 1

A treated solution of Comparative Example 1 was obtained as in Example 1 except for the following points. The PBS placed in the space where the counter electrode was placed and the raw material solution of Example 1 were each stirred with a magnetic stirrer at a temperature in the range of 15° C. to 25° C. for 16 hours without applying voltage between the working electrode and the counter electrode. The fructose concentration in the treated solution of Comparative Example 1 was measured with Fructose Colorimetric/Fluorometric Assay Kit available from Sigma-Aldrich. The result is shown in FIG. 7.

FIG. 7 is a graph showing changes in fructose amount in Comparative Example 1. In FIG. 7, the vertical axis indicates the fructose concentration. As shown in FIG. 7, the fructose concentration in the treated solution of Comparative Example 1 was 320 mM. It is considered that about 8% of fructose in the raw material solution was converted due to the enzymatic reaction.

NADH in the raw material solution acts as a coenzyme of the MDH and is oxidized into NAD⁺ along with the enzymatic reaction involving MDH. In addition, it is considered that, due to this enzymatic reaction, fructose is reduced into mannitol.

According to Example 1, when voltage is applied between the working electrode and the counter electrode, NAD⁺ generated by the enzymatic reaction is reduced into NADH and is thus regenerated due to migration of electrons from the working electrode. The regenerated NADH again acts as a coenzyme of MDH and is oxidized into NAD⁺ while fructose is reduced to mannitol. As such, in Example 1, it is considered that fructose, which had been contained in high concentration in the raw material solution, was converted into mannitol.

In contrast, according to Comparative Example 1, it is considered that, since voltage is not applied between the working electrode and the counter electrode, NAD⁺ generated by the enzymatic reaction is not reduced into NADH.

Thus, according to the features of the present disclosure, fructose can be converted with high efficiency.

According to the present disclosure, for example, fructose contained in food can be highly efficiently converted into mannitol.

What is claimed is:

1. A fructose decomposition method comprising:
bringing a liquid containing mannitol dehydrogenase, fructose, and a coenzyme into contact with a working electrode to electrochemically reduce the coenzyme and decompose the fructose by the mannitol dehydrogenase.

2. The fructose decomposition method according to claim 1, wherein
the coenzyme is oxidized as mannitol is generated from the fructose by the mannitol dehydrogenase, and
the coenzyme is reduced by receiving electrons from the working electrode.

3. The fructose decomposition method according to claim 1,
wherein the coenzyme is nicotinamide adenine dinucleotide.

4. The fructose decomposition method according to claim 1,
wherein voltage is applied to the working electrode for a particular period of time.

5. The fructose decomposition method according to claim 1,
wherein voltage is applied to the working electrode until a current value in the working electrode has reached a particular value or lower.

6. A fructose decomposition device comprising:
a working electrode;
a counter electrode; and
a voltage application device for applying voltage between the working electrode and the counter electrode,
wherein the fructose decomposition device electrochemically reduces a coenzyme by applying the voltage and decomposes fructose by mannitol dehydrogenase.

7. The fructose decomposition device according to claim 6,
wherein the working electrode supplies electrons to the coenzyme by applying the voltage.

8. The fructose decomposition device according to claim 6,
wherein the coenzyme is nicotinamide adenine dinucleotide.

9. The fructose decomposition device according to claim 6,
wherein the voltage application device keeps applying the voltage for a particular period of time.

10. The fructose decomposition device according to claim 6,
wherein the voltage application device keeps applying the voltage until a current value in the working electrode has reached a particular value or lower.

11. A mannitol production method comprising:
bringing a liquid containing mannitol dehydrogenase, fructose, and a coenzyme into contact with a working electrode to electrochemically reduce the coenzyme and convert the fructose into mannitol by the mannitol dehydrogenase.

12. A mannitol production device comprising:
a working electrode;
a counter electrode; and
a voltage application device for applying voltage between the working electrode and the counter electrode,
wherein the mannitol production device electrochemically reduces a coenzyme by applying the voltage and converts fructose into mannitol by mannitol dehydrogenase.
